(19) United States
(12) Patent Application Publication
(10) Pub. No.: US 2010/0292272 A1
(43) Pub. Date: Nov. 18, 2010

(54) SUPRAMOLECULAR CO-COLLOIDS PRODUCED USING MACROCYCLIC POLYANIONIC SYSTEMS

(76) Inventors: Anthony William Coleman, Caluire (FR); Adina Nicoleta Lazar, Valcea (RO); Jean-Yves Pierre Paul Renault, Sceaux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/377,065

(22) PCT Filed: Aug. 10, 2007
(86) PCT No.: PCT/FR07/01363

§ 371 Date: May 24, 2010

(30) Foreign Application Priority Data
Aug. 11, 2006 (FR) 0607306

(51) Int. Cl. A61K 9/10; A61K 47/10; A61K 31/337; A61K 31/454; A61P 35/04; A61P 27/02

(52) U.S. Cl. 514/323; 514/772; 514/449

(57) ABSTRACT

The present invention relates to a method preparing co-colloidal dispersions of active hydrophobic substances, such as pharmaceutical products, products having cosmetic properties or any other chemical product. The co-colloidal dispersions thus obtained are characterized in that they are formed by amphiphilic complexes resulting from combinations by non-covalent bonds between a hydrophobic active substance and a suitable hydrophilic molecule.

The present invention describes a method for dispersing hydrophobic substances in aqueous phase. Such a method is useful for multiple applications, notably for the formulation of active pharmaceutical substances or cosmetics.

One of the major issues met during the development of biologically active substances lies in their hydrophobic nature. In aqueous phases, active substances tend to precipitate, thereby considerably limiting their bioavailable concentrations and their biological activity. It is possible to increase the bioavailable concentrations of active hydrophobic substances by using different means, for example, by inclusion in suitable molecules, such as cyclodextrins, or by encapsulation in an suitable colloidal dispersion, such as micelles, liposomes, or lipidic nanoparticles. The encapsulation methods can also protect the molecules from degradation by light or by enzymatic reactions. The other methods that allow modification of the concentration of active substances are the use of co-solvents or co-solutes or pH modifications. Besides, the use of co-crystals allows modifications of the dissolution kinetics and thereby to modify the pharmacokinetics profile of an active substance.

To prepare colloidal dispersion, one uses one or several amphiphilic molecules. An amphiphilic molecule is a molecule that combines covalently a hydrophobic apolar group and a hydrophilic polar group. Amphiphilic molecules have a double affinity, for the apolar phases (air, oil, organic solvents) on one hand, and for water, on the other hand. Lipids are examples of amphiphilic molecules.

Above a critical concentration in an aqueous environment, amphiphilic molecules are generally capable to auto-assemble by forming colloidal structures that are characterized by particles in suspension. The type of colloidal structure that is obtained depends on the chemical structure of the amphiphilic molecules, and notably on the ratio between the sizes of the polar head and apolar tail of the molecule.

Colloidal dispersions can be used to increase the bioavailable concentrations of hydrophobic active substances. The principle is that the active hydrophobic substance should be associated with the hydrophobic groups of the amphiphilic molecules.

However, one drawback of the methods that use amphiphilic molecules lies in the fact that the maximum percentage (also called charge factor, calculated in weight units) of active substance that can be dispersed in the colloidal structure is too low, approximately 5 to 10%. Another drawback of these colloidal assemblies is their low stability during time, notably at room temperature.

These problems have been overcome by using solid lipid nanoparticles (SLN) that include nanocapsules and polymeric nanospheres. Such structures are matrix structures. In this case, the charge factors are generally higher, approximately 30%, and the stability during time is improved.

Besides, methods that use calixarenes products have been described. The US patent 2005/0240051 A 1 authored by N. Yasuda et M. Furukawa describes a method that uses calixarenes to solubilize carbon-based materials, such as fullerenes, graphite, diamond and stains, in a non-aqueous organic solvent such as toluene, an oil or a resin. However, the calixarenes used in this patent are not soluble in water and this patent is not related to a dispersion method for pharmaceutical or cosmetic substances.

In the patent application WO 03/024583, the authors describe a system for dispersion in water that uses amphiphilic and non-hydrophilic calixarenes. These amphiphilic calixarenes can spontaneously auto-assemble and form colloidal dispersions. The addition of a hydrophobic molecule to this colloidal calixarenes suspension is possible but is not necessary.

In two articles (Eur. J. Pharm. Biopharm., 2004, 58(3), 629-636 and J. Pharm. Pharmacol., 2004, 56(6), 703-708), W. Yang and M M. De villiers have shown that it was possible to solubilize nifedipine, furosemide and niclosamide (active substances of pharmaceutical interest that are poorly soluble in water) by using para-sulfonato-calixarenes in an aqueous acidic solution. However, the objectives of this work and the processes that are used are different from the invention described in the present patent. Indeed, W. Yang and M M. De villiers describe only a solubilization method and not a colloidal dispersion method, that is notably characterized by the presence of particles.

More recently, the co-crystal systems, based on supramolecular non-covalent assembly systems, have been used to modify the pharmacokinetic and physical properties of pharmaceutical products. In this case, one uses the known non-covalent interactions so that a crystallizing substance is bound to a pharmaceutical substance, thereby providing different physical properties, stability and dissolution rate.

In view of the previous information, the purpose of the invention is notably to implement a new method that allows the dispersion of hydrophobic active substances, such as substances of pharmaceutical or cosmetical interest, in aqueous solution.

The purpose of the invention is also to propose new dispersions that do not have the drawbacks mentioned in the previous art and, in particular, that are stable in time.

Finally, the purpose of the invention is also to propose new dispersions which can be used as vehicles for hydrophobic active substances of pharmaceutical or cosmetic interest.

In a surprising and advantageous way, the applicant has now just discovered that these purposes could be reached by implementing a process of dispersing a hydrophobic molecule, such as a hydrophobic active pharmaceutical or cosmetic substance of interest, in an aqueous phase, which includes a step consisting of forming a supramolecular complex between the said hydrophobic molecule and the said hydrophilic molecule.

In the following text, the dispersions obtained within the framework of the present invention are identified by the term “co-colloidal dispersions.”

So, the process of dispersion according to the present invention, allows generation of co-colloidal dispersions of hydrophobic active molecules in aqueous solution.

Furthermore, these co-colloidal dispersions are stable with time.

So, in a first version of the invention, a co-colloidal dispersion in an aqueous medium of at least one supramolecular amphiphilic complex, in which the said amphiphilic complex includes one hydrophilic molecule and one hydrophobic molecule that are associated by non-covalent bounds, is proposed.

The present invention uses the formation of an inter-molecular and non-covalent association between a molecule of a hydrophobic active substance, termed an invited molecule, and an suitable hydrophilic host molecule. This association results in the formation of supramolecular amphiphilic complexes combining a polar head and an apolar tail. In aqueous phases, these supramolecular amphiphilic complexes are capable of auto-assembling and forming particles suspensions that characterize colloidal dispersions. The size of the obtained particles ranges between 10 nm and 1 μm, and preferentially between 100 and 450 nm.

Therefore, this method allows dispersion of a hydrophobic molecule in an aqueous phase in the form of a dispersion of a colloidal nature. An aqueous phase contains at least 50% of water and can be constituted, for example, by pure water, by an isotonic solution, by a physiological medium or by a pharmaceutical solution for injection or for topical use.

The co-colloidal dispersions described in the present invention are different from all the known colloidal dispersions, for example those that use lipids, because there are no covalent bounds in the assembly of the amphiphilic entity. They are also different from all the known colloidal dispersions because the hydrophobic molecule that one wants to disperse is necessary to form the colloidal structure in the aqueous medium.

Thus, contrary to the colloidal dispersions described in the patent application WO 03/024583, the co-colloidal dispersions described in the present invention are constituted by the assembly of two molecules, one being hydrophilic and the other being hydrophobic forming an amphiphilic supramolecular complex; thus, none of the two molecules can form a colloidal dispersion when used alone. It is indeed a different state of matter.

The host hydrophilic molecules that are used in the invention include notably products from the calixarene family and preferentially anionic hydrosoluble calixarenes, for example the para-sulfonato calix[4]arene and the para-sulfonato calix[6]arene molecules.

In the sense of the present invention, an hydrophilic substance is a compound that is soluble in water and that is able to create hydrogen bonds with water molecules.

In particular, the calixarenes used in the present invention are hydrophilic compounds because they bear polar and ionizable functions.

The calixarenes used in the present invention have the general structure (I):

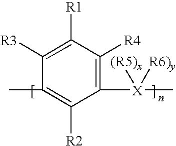

in which,
R₁ represents an hydrogen atom or a polar group, such as an hydroxyl, a carboxylate, a sulfonate, a phosphonate, a sulfonamide, or an amide, or an alkyl, alkene or alkyne group, linear, ramified or cyclic, eventually substituted, notably by a polar group, and preferentially an hydrogen atom or a sulfonate group.
R₂ represents a polar group, such as an hydroxyl, a carboxylate, a sulfonate, a phosphonate, a sulfonamide, an amide, an ester or an alkyl, ramified or substituted by a polar group, and preferentially an hydroxyl if R₁ is a sulfonate or a phosphonate group if R₁ is an hydrogen atom.
R₃, R₄, R₅, R₆ represent each and independently an hydrogen atom, an alkyl, alkene or alkyne group, eventually substituted, notably by a polar group, in particular hydroxyl functions, substituted or not, and preferentially an hydrogen atom.
X represents an atom of carbon, (when x=1 and y=1), of sulfur or oxygen (when x=0 and y=0) or of nitrogen (when x=1 and y=0);
n is a number between 4 and 8.

The invited hydrophobic active substances that can be dispersed in an aqueous phase according to the invention are numerous. The examples described below show that the invention allows dispersion in the form of co-colloidal dispersion molecules that have very different chemical structures. The experiments that were conducted show that the molecules to be dispersed should present an hydrophobic part and chemical groups which allow for the non-covalent interactions with the hydrophilic host molecules. The non-covalent bonds that are implemented can include hydrogen bonding, ion-ion interactions, ion-dipole interactions, cation-π interactions, π-π interactions, Van Der Walls forces or hydrophobic interactions. The size of the hydrophobic active substance is not a restrictive condition.

Thus, the purpose of the present invention is also a dispersion process in an aqueous medium of a hydrophobic molecule of pharmaceutical interest that comprises a step during which a supramolecular amphiphilic complex between the said hydrophobic molecule and a hydrophobic molecule is formed.

As a variation, a dispersion process in aqueous medium of a hydrophobic cosmetic substance that comprises a step during which a supramolecular amphiphilic complex between the said hydrophobic substance and a hydrophobic molecule is formed.

The present invention also concerns a process that allows the obtention of co-colloidal dispersions which consists in adding a composition comprising, in an organic solvent, at least one anionic hydrosoluble calixarene to a composition comprising, in an organic solvent, a hydrophobic molecule, in adding an aqueous solvent, and in eliminating the said organic solvent.

In particular, the process allowing the formation of co-colloidal dispersions is the following.

1.) Molecules Solubilisation in Organic Solvent.

The host molecule and the invited molecule are beforehand solubilized separately in a powerful organic solvent, preferentially tetrahydrofurane (THF). The products quantities and the solvent volumes are determined according to the final concentrations and volume that are wished. For example, at the laboratory scale, to obtain a co-colloidal dispersion of active substance at a final concentration of 50 mg/L, 2.5 mg of host molecule, preferentially para-sulfonato calix[4]arene, are solubilized in 5 mL of THF and 2.5 mg of active substance are separately solubilized in 5 ml of THF.

2.) Co-Colloïd Formation.

Then, the two solutions are mixed at equal volumes (5 mL for a preparation at laboratory scale) and maintained under constant agitation, for example with a magnetic stirrer set at 350 rounds per minute or with a Vortex type equipment. Then, the final solvent, for example pure water (50 mL) is progressively added at a fixed rate of 200 mL/s and with maintaining constant agitation during a half hour.

3.) Co-Colloid Formation.

Thereafter, the initial organic solvent is eliminated, preferentially by evaporation, for example by placing the dispersion during 15 minutes, under reduced pressure and at a temperature of 40° C. The co-colloidal dispersion that is finally obtained appears as homogenous, slightly opaque at visual examination, thereby showing that the hydrophobic active substance is dispersed homogenously.

The technical conditions (solvent volumes, product quantities, duration of each phase, etc.) indicated above are given as example and can, naturally, be adapted according to the nature of the hydrophobic active substance, as well as the wished final volumes and concentrations.

The active substances which can be dispersed according to the invention include for example substances that are active on the peripheral and central nervous system, on the renal, cardiovascular, gastro-intestinal, blood, immune, hormonal, genital or reproductive functions, anti-inflammatory, anti-parasitic, antibiotics, anticancer products, antidotes, vitamins, products for the parentérale nutrition or products for dermatologic, topical or ophthalmologic use.

The hydrophobic active substances of pharmaceutical interest which can be dispersed under the form of co-colloids include for example penclomedine, tamoxifene, tetracaine, chlorhexidine, mifepristone, etoposide, clarithromycine, benzafibrate, azithromycine, itraconazole, propofol, rhizoxin palmitoyl, clofibrate, clofibric acid, gemfibrozil, acediasulfone, acetophenetidine, 1-acetyl-2-phenylhydrazine, alexidine, ambenomium chloride, amidinomycine, p-chlorobenzhydrazide, 2,3-diaminophenazine, dichlorphenamide, divicine, etc.

In the sense of the present invention, a hydrophobic substance is an active substance whose solubility in water is not sufficient to prepare formulations at concentrations that are sufficiently high to obtain the desired activity.

Preferentially, the hydrophobic active substances of pharmaceutical interest that can be dispersed as co-colloids include tamoxifen, tetracaine, chlorhexidine, mifepristone, thalidomide, and the molecules of the taxane family.

The taxanes constitute a group of pharmaceutical products, including notably docetaxel and paclitaxel, that are used for the treatment of cancer. The taxanes have the property to stop the growth of cancer cells by interfering with cellular structures, called microtubules, that play an essential role for cell division.

Microtubules are formed when cells start to divide and are destroyed after cell division. Taxanes prevent the microtubules destruction and thus prevent cell division.

Docetaxel and paclitaxel are administered to patients by the intravenous route for the treatment of cancer diseases, notably lung, prostate, ovary or breast cancers.

Docetaxel and paclitaxel are molecules with low water solubility. Thus, the preparation of a pharmaceutical formulation with docetaxel generally requires to, firstly solubilize docetaxel in a mix of ethanol and polysorbate, then to dilute this solution in an aqueous solution. This transparent solution is then injected as an intravenous perfusion.

However, the presence of residual polysorbate in such a solution can lead to significant toxicity effects during the administration of this said solution by the intravenous route. These toxic effects can necessitate to pre-treat the patients with an anti-inflammatory drug.

Thus, the process according to the invention presents the advantage to disperse docetaxel in an aqueous medium without using polysorbate. Therefore, one obtains a less toxic pharmaceutical formulation that can be administered by the intravenous route.

Besides, when the drug under the form of a co-colloidal dispersion in aqueous medium of at least one amphiphilic complex comprising at least one anionic hydrosoluble calixarene and docetaxel is administered, this drug present the additional advantage to produce a satisfactory anti-cancer effect.

Therefore, the present invention consists in an anti-cancer drug under the form of a co-colloidal dispersion in aqueous medium of at least one amphiphilic complex comprising at least one anionic hydrosoluble calixarene and one molecule of the taxane family.

In particular, the purpose of the present invention is a pharmaceutical drug under the form of a co-colloidal dispersion in aqueous medium of at least one amphiphilic complex comprising at least one anionic hydrosoluble calixarene and docetaxel.

More particularly, the invention relates to a pharmaceutical drug under the form of a co-colloidal dispersion in aqueous medium of at least one amphiphilic complex comprising at least one anionic hydrosoluble calixarene and docetaxel for use by the intravenous route to treat cancer diseases.

In particular, this pharmaceutical drug is used to treat breast and lung cancer.

Docetaxel can be present in the co-colloidal dispersion at a concentration ranging from 0.01% to 1% in weight, relatively to the total weight of the dispersion.

Besides, thalidomide is a molecule known for its anti-angiogenic properties. These properties allows the use of thalidomide as a pharmaceutical drug to treat a specific type of cancer known as multiple myeloma.

Thalidomide is a pharmaceutical drug that is generally administered by the oral route. Besides, there are no pharmaceutical formulations allowing administration by the parenteral route.

Thus, the process according to the invention present the advantage to disperse thalidomide in aqueous medium. Thus, one obtains a pharmaceutical formulation that can be administered by the parenteral route, for example by the intravenous, intramuscular, subcutaneous or intravitreal route.

Therefore, the purpose of the present invention is also a pharmaceutical drug under the form of a co-colloidal dispersion in aqueous medium of at least one amphiphilic complex comprising at least one anionic hydrosoluble calixarene and thalidomide.

In particular, this pharmaceutical drug can be administered to cancer patients and for whom the oral administration is difficult or impossible.

Indeed, some patients present with lesions of the buccopharyngeal region or the oesophagus that make the oral administration of pharmaceutical drugs very difficult. In this case, thalidomide can be administered by the parenteral route, and notably by the intravenous route.

In particular, thalidomide can be administered to patients by the intravitreal route to treat age-related macular degeneration (AMD.)

AMD is disease of the retina that is caused by a progressive degeneration of the macula, the central part of the retina, that appears most often from the age of 50 years, and more frequently from the age of 65 years, provoking an important decline of visual capacity, but not a complete loss.

AMD is characterized by the appearance of choroïdal neovessels (new blood vessels). They develop either under the pigmented epithelium and thus are designated as “occult” or above the epithelium and thus are designated as “visible.” Because of its anti-angiogenic properties, thalidomide can prevent the development of new vessels and thus stabilize the progression of the disease.

The present invention relates more particularly to a pharmaceutical drug under the form of a co-colloidal dispersion in aqueous medium of at least one amphiphilic complex comprising at least one anionic hydrosoluble calixarene and thalidomide for its use by the intravitreal route to treat age-related macular degeneration.

Thalidomide can be present in the co-colloidal dispersion at concentrations ranging from 0.01% to 1% in weight, relatively to the total weight of the dispersion.

Co-colloidal dispersions can also contain miscellaneous additives such as osmotic pressure regulators, for example sucrose or glycerine, oxidants such as alpha-tocopherol or ascorbic acid or preservatives such as methyl, ethyl- and butyl-paraben.

The compositions for cosmetic use prepared according to the invention include preparations for skin or hair, such as shampoos, preparation for use on skin or lotions for sun protection.

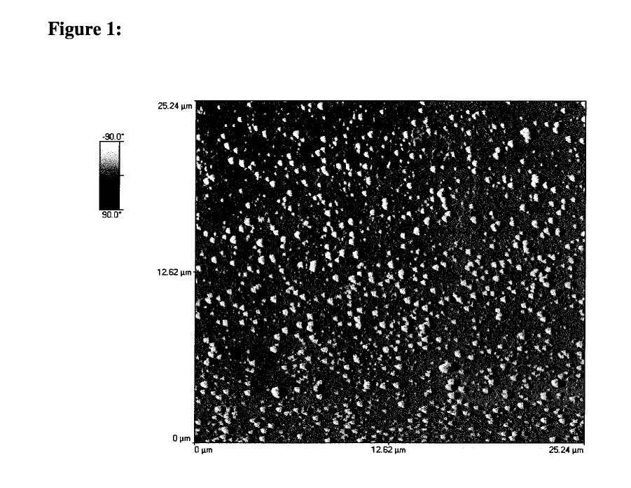

The following examples are intended to better understand the invention without presenting a limitating character. These examples are illustrated by FIGS. 1-4 in annex that present co-colloidal dispersions which are consistent with the present invention and which were characterized by means of various analysis techniques.

### EXAMPLES

### Préparation of Co-Colloidal Dispersions

The examples described below allowed to characterize the method of preparation of the co-colloidal dispersions as well as to characterize the quality the said dispersions.

One prepares a co-colloidal dispersion according to the above-mentioned process. Thus, in a first solution, 2.5 mg of para-sulfonato calix[4]arene (designated as C4S in the tables below) are solubilized in 5 mL of THF and, in a second solution, 2.5 mg of tamoxifen (anti-cancer drug) are solubilized separately in 5 mL of THF. Tamoxifen is a hydrophobic active substance.

Thereafter, the two solutions are mixed at equal volumes and maintained under constant agitation with a magnetic stirrer set at 350 rounds per minutes or a Vortex type equipment. Then, 50 mL of pure water are progressively added at a fixed rate of 200 mL/s while maintaining constant agitation during a half hour.

Thereafter, THF is evaporated by placing the mixed solution during 15 minutes, under reduced pressure and at a temperature of 40° C.

Following this process, a co-colloidal dispersion is obtained with a final concentration of tamoxifen and C4S of 50 mg/mL. The co-colloidal dispersion appears homogenous at visual examination, thereby indicating that tamoxifen was dispersed homogenously.

This process was done three times by using para-sulfonato calix[6]arene (designated as C6S in the tables below) and tamoxifen at different concentrations, so as to obtain co-colloidal dispersions with different final concentrations of para-sulfonato-calix[6]arene and tamoxifen.

This process was also done four times with using para-sulfonato calix[4]arene and tetracaine at different concentration so as to obtain final co-colloidal dispersions having different final concentrations of para-sulfonato calix[4]arene and tetracaine (local anesthetic).

This process was done twice with using para-sulfonato calix[6]arene and para-sulfonato calix[4]arene with chlorhexidine (antibiotics) at similar concentrations.

This process was done twice with using para-sulfonato calix[6]arene and para-sulfonato calix[4]arene with mifepristone (abortifacient) at similar concentrations.

Tamoxifen, tetracaine, chlorhexidine and mifepristone used in these preparations are pharmacologically-active substances known for their weak solubility in water.

The following table indicates the co-colloidal dispersions that were obtained, as well as the final concentrations in host molecules and in hydrophobic active substances (invited molecules) present in these dispersions.

| Co-colloidal | Host molecule | Invited molecule |
|---|---|---|
| dispersion no. | (final concentration) | (final concentration) |
| 1 | C4S (50 mg/l) | Tamoxifen (50 mg/l) |
| 2 | C6S (50 mg/ml) | Tamoxifen (50 mg/l) |
| 3 | C6S (55 mg/ml) | Tamoxifen (25 mg/l) |
| 4 | C6S (87.4 mg/ml) | Tamoxifen (12.4 mg/l) |
| 5 | C4S (50 mg/l) | Tetracaine (50 mg/l) |
| 6 | C4S (70 mg/l) | Tetracaine (30 mg/l) |
| 7 | C4S (83 mg/l) | Tetracaine (17 mg/l) |
| 8 | C4S (50 mg/l) | Tetracaine (50 mg/l) |
| 9 | C4S (50 mg/l) | Chlorhexidine (50 mg/l) |
| 10 | C6S (50 mg/l) | Chlorhexidine (50 mg/l) |
| 11 | C4S (50 mg/l) | Mifepristone (50 mg/l) |
| 12 | C6S (50 mg/l) | Mifepristone (50 mg/l) |

In all cases, a homogenous, slightly white, co-colloidal dispersion was obtained. Thus, the hydrophobic active molecules were dispersed in an aqueous phase while they are little or weakly soluble in water. Using this method, these co-colloidal dispersions can be used as vehicles for active hydrophobic substances of pharmaceutical interest.

Different techniques were then used to characterize a co-colloidal dispersion having a final concentration in tamoxifen and in para-sulfonato calix[6]arene equal to 50 mg/mL (corresponding to the example no. 2 in the above table). In particular, dynamic light scattering, atomic force microscopy, scanning electronic microscopy and transmission electronic microscopy are used. These techniques allow the analysis of the particles in suspension in aqueous phase or after drying.

Dynamic Light Scattering (DLS) allows the obtention of information regarding article sizes. When a monochromatic and polarized light beam hits a particle, the light is diffused in all directions in space. The variations in intensity of the diffused light are associated with the diffusion rate of the molecules in the studied region, because they are animated by brownian movements. Data are directly analyzed to provide diffusion coefficients. When several molecular species are present, a distribution of diffusion coefficients can be observed. Thereafter, these data are treated to obtain the particles diameters. Indeed, the relation between the diffusion coefficient and the size is based on the theoretical relations of the brownian movements of spherical particles (Stockes-Einstein law). Thus, for a medium with known viscosity, the measure of the distribution coefficients is sufficient to calculate the particles hydrodynamic radius. The measurement of the size of particles in solution is done at room temperature. The analyses are carried out using a 4700C MALVERN spectrometer. The light source is a SIEMENS 40 MW laser. Each measurement is repeated ten times, the sizes and the reported polydispersity indices correspond to the mean of the ten measurements.

Observation by Atomic Force Microscopy (AFM) consists in a surface analysis, by mean of a very fine point of a few micrometers long and one hundred of Angströms of diameter, that is set up at the extremity of mobile arm made of silicium, called a microcantilever, and having a known force. The different marketed equipments present with various geometries. The AFM that was used is based on the Explorer technique (Topometrix Inc.). In this set up, the piezo-electrical ceramics that is used to generate the scanning movement holds the micro-cantilever. With the other techniques, that are more commonly used, the ceramics holds the sample and the AFM head stays without movements. The equilibrium of forces between the surface of the sample and the point induces modifications in the positions of the microcantilever. The signal is recorded on a photo-detector with four quarters via the reflexion of a laser beam on the microcantilever which deflection is proportional to the forces acting on the probe and is thus measured. Thereafter, these data are transformed into spatial coordinates, thereby generating a surface image. AFM measures the forces between the point and the sample. These forces depend on the nature of the sample and on the distance between the point and the sample. When the point approaches the surface, it is submitted firstly to forces that are attractive at long distances (van der Waals forces). Thereafter, when approaching more the surface, the electronic orbitals of the point and of the sample generate repulsive forces that neutralize the attractive forces before becoming the dominant forces. The measurements are done by using an “Explorer ThermoMicroscope” microscope that is equipped with a 100 μm scanner in non-contact mode. The scanning speed is 1-2 Hz. For each sample, the scannings are done at 50 μm, 20 μm, 10 μm and 5 μm. The microcantilevers are made of silicium, the resonance frequency f₀ is 260 kHz and the stiffness constant is 45 N.

For the studies done with scanning electronic microscopy, a 50 μL sample of co-colloidal dispersion is placed on glass strip, then the sample is allowed to dry at room temperature during 18 hours. The samples are then covered with a gold-palladium layer and observed with a Hitachi S800 electronic microscope at 15 kV.

Some studies that use transmission electronic microscopy after freeze fracture and after negative staining are done. For these studies, a 5 μL sample of co-colloidal dispersion at 0.2 mM on a copper grid (300-mesh) covered with a Formvar® film (Polyvinyl formal). After 5 minutes adsorption, the samples are negatively stained, either with a sodium silicotungstate aqueous solution at 1%, or with an uranyl acetate solution at 4%. They are immediately observed with a Philips CM120 electronic microscope at 80 kV.

The co-colloidal dispersion according to example no. 2 was studied as follows:

Atomic Force Microscopy (AFM) Observation

FIG. 1 shows the image obtained in microscopy of a co-colloidal dispersion that was made with tamoxifen and para-sulfonato calix[6]arene C6S according to example no. 2.

Thus, the AFM studies show the existence of nanoparticles, even after drying of the sample.

A topographical analysis of the size of these particles reveals an height of 75 nm and a diameter of approximately 300 nm. This diameter is slightly greater that the diameter determined by dynamic light scattering diffusion (see results above) which is explained by the slight flattening of particles after drying of the sample

Transmission Electronic Microscopy Observations

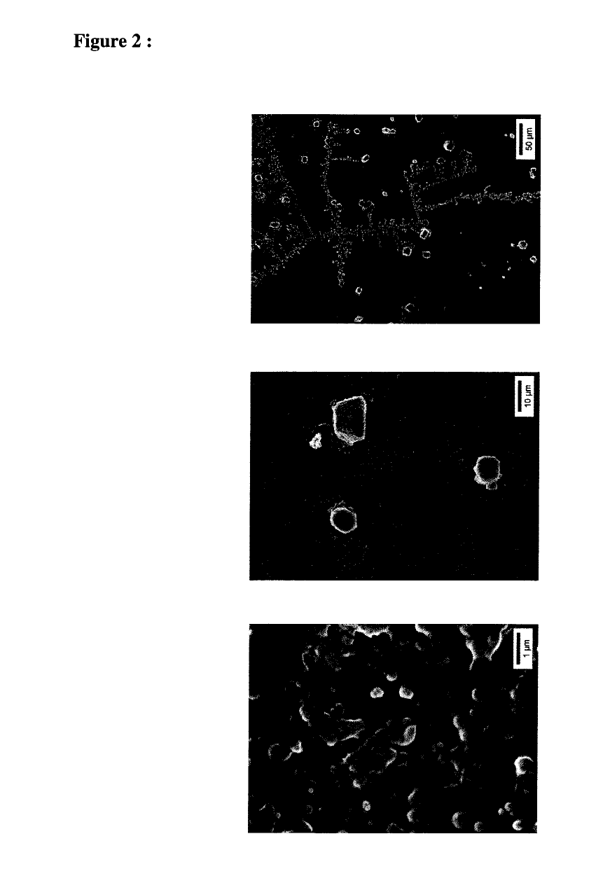

FIG. 2 shows images of a co-colloidal dispersion according to example no. 2 using scanning electronic microscopy with three different magnifications.

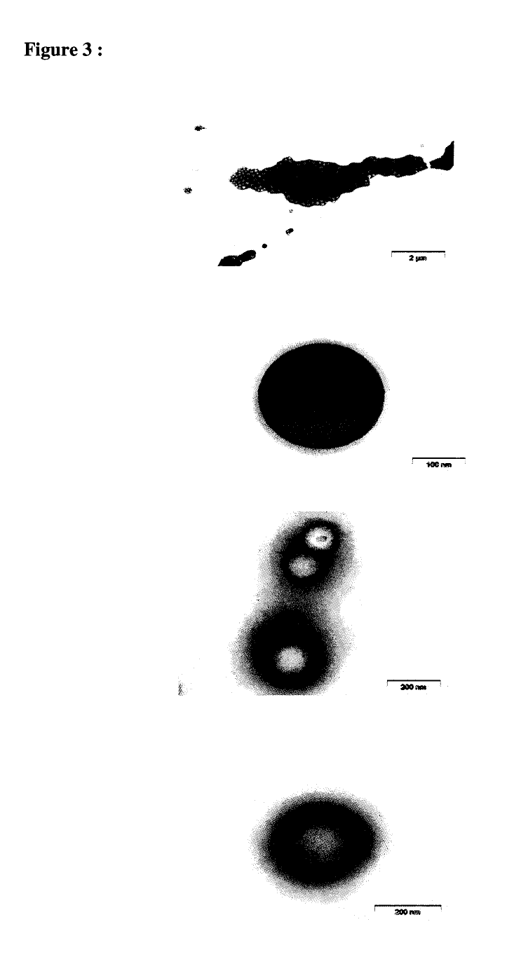

FIG. 3 shows images of a co-colloidal dispersion according to the example no. 2 using transmission electronic microscopy after negative staining with three different magnifications.

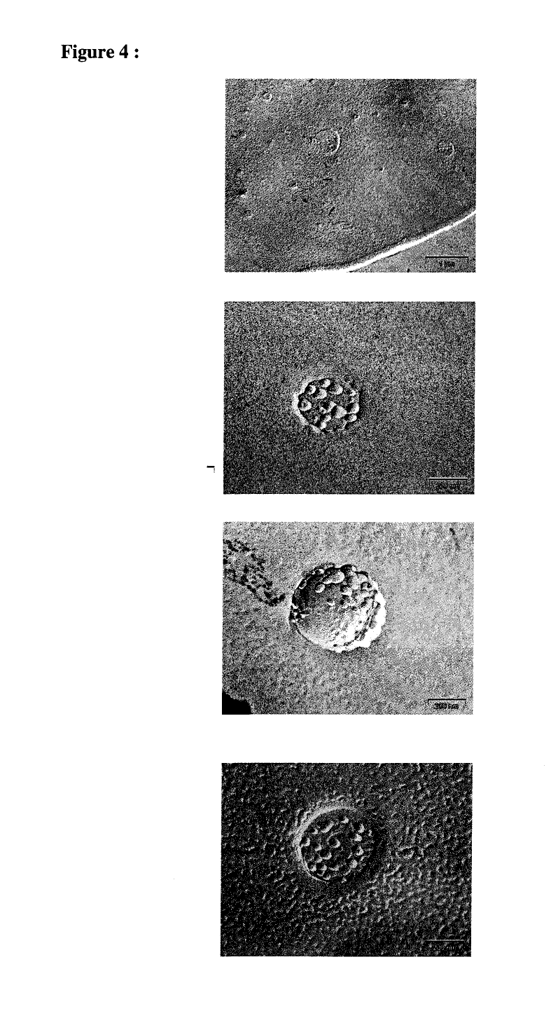

FIG. 4 shows images of a co-colloidal dispersion made with tamoxifen and para-sulfonato calix[6]arene using transmission electronic microscopy after freeze drying fracture and with three different magnifications.

These images allow identification the particles in the co-colloidal dispersion. They show notably a spherical structure, but also an internal structure organized in small vesicles. Such a structure is different from the multi-lamellar structure of liposomes.

Besides, similar images were obtained with co-colloidal dispersions made with para-sulfonato calix[6]arene and griseofulvin or chlorhexidine.

Measurement of the Particles Size and Polydispersity

The co-colloidal dispersion made with tamoxifen and para-sulphonato calix[6]arène according to the example no. 2 was analyzed using dynamic light scattering immediately after its formation.

The measurements were repeated ten times and show the existence of a co-colloidal dispersion constituted with mono-dispersed particles having a mean diameter of approximately 230 nm. The polydispersity index is 0.03 thereby demonstrating that the particles have a constant diameter.

These measurements were repeated ten times on the co-colloidal dispersion according to the examples 1 and 3 to 12. The results are grouped in the following table.

| | | Particles average |
|---|---|---|
| Dispersion | Host molecule (final concentration)/ | measurement |
| no. | Invited molecule (final concentration) | By DLS |
| 1 | C4S (50 mg/l)/Tamoxifen (50 mg/l) | 190 nm |
| 2 | C6S (50 mg/ml)/Tamoxifen (50 mg/l) | 230 nm |
| 3 | C6S (55 mg/ml)/Tamoxifen (25 mg/l) | 225 nm |
| 4 | C6S (87.4 mg/ml)/Tamoxifen (12.4 mg/l) | 215 nm |
| 5 | C4S (50 mg/l)/Tetracain (50 mg/l) | 205 nm |
| 6 | C4S (70 mg/l)/Tetracain (30 mg/l) | 440 nm |
| 7 | C4S (83 mg/l)/Tetracain (17 mg/l) | 269 nm |
| 8 | C4S (50 mg/l)/Tetracain (50 mg/l) | 220 nm |
| 9 | C4S (50 mg/l)/Chlorhexidine (50 mg/l) | 275 nm |
| 10 | C6S (50 mg/l)/Chlorhexidine (50 mg/l) | 460 nm |
| 11 | C4S (50 mg/l)/Mifepristone (50 mg/l) | 238 nm |
| 12 | C6S (50 mg/l)/Mifepristone (50 mg/l) | 191 nm |

These results show that the preparation method for co-colloidal dispersions allow for obtaining populations of particles that are relatively homogenous with sizes ranging approximately between 150 and 450 nm, according to the nature of the host molecules and of the invited molecules and of the concentrations ratio between the two molecules.

Stability Study of Co-Colloidal Dispersions

The stability of co-colloidal dispersions according to the examples 1 to 12 was studied for temperatures of 4, 20, 40 and 80° C. The dispersions are placed in incubators with controlled temperatures and samples are periodically taken to analyze the size and the polydispersity of the particles or the morphology by atomic force microscopy. The results are grouped in the following table.

| Dispersion | Host molecule | Particles stability at different temperatures as |
|---|---|---|
| no. | Invited molecule | measured by dynamic light scattering |
| 1 | C4S (50 mg/l) | At 20° C., the mean particle size slightly decreases from 190 |
| | Tamoxifen (50 mg/l) | to 130 nm over a 15-day period. At 80° C., the particles |
| | | mean size decreases from 190 to 175 nm over a 15-day |
| | | period |
| 2 | C6S (50 mg/ml) | At 4° C., 20, 40 and 80° C., the mean particle size is |
| | Tamoxifen (50 mg/l) | maintained close to 200 nm over a 43-day period |
| 5 | C4S (50 mg/l) | At 4° C., the mean particle size increases from 205 to 230 nm |
| | Tetracain (50 mg/l) | and over a 15-day period, then is maintained stable |
| | | during 28 days. At 20, 40 and 80° C., the mean particle size |
| | | decreases from 205 to 165 nm and over a 15-day period, |
| | | then is maintained stable during 28 days. |
| 8 | C4S (50 mg/l) | At 4 and 20° C., the mean particle size decreases from 220 to |
| | Tetracaine (50 mg/l) | 179 and 150 nm, respectively, over a 15-day period |
| 11 | C4S (50 mg/l) | At 20 and 80° C., the mean particle size decreases from 238 |
| | Mifepristone (50 mg/l) | to 220 nm over a 15-day period |
| 12 | C6S (50 mg/l) | At 20° C., the mean particle size decreases from 190 to 174 nm |
| | Mifepristone (50 mg/l) | over a 15-day period. At 80° C., the mean particle size is |
| | | maintained at 190 nm over 15 days. |

These results show that the particle size varies slightly, even when the preparations are maintained during several weeks at low temperatures (4° C.) or high temperatures (80° C.), thereby showing the remarkable stability of co-colloidal dispersions.

Thus, the results show that the particle size is almost uninfluenced by the temperature used for storage. Similar results were observed for other co-colloidal dispersions that are not shown as examples.

Study of the Interactions Between Co-Colloidal Dispersions and Albumin

In order to envisage an intravenous administration of co-colloidal dispersions, it is necessary to ensure that such dispersions do not aggregate when in contact with albumin, which is the most abundant protein in the blood system. Thus, the interaction of co-colloidal dispersions with albumin was also studied. A solution of bovine serum albumin (BSA) at 1 mg/mL was added to a co-colloidal dispersion made with tamoxifen and the mixed solution was analyzed by dynamic light scattering and by atomic force microscopy.

The analyses by light scattering show that the particles size with BSA are greater than 1 μm, but they stay monodispersed. This phenomenon can be explained by the presence of a protective matrix of proteins that surrounds the colloidal particles. On the contrary, the images obtained by atomic force microscopy reveal the existence of particles with sizes that are much smaller than the normal particles size (15 nm instead of 200 nm or 1000 nm). This can be explained by the fact that BSA forms a relatively dense protein gel that covers the co-colloidal particles and let see only a part of the particles at the surface of the area examined by atomic force microscopy. Thus, the interactions with the major blood protein do not change the co-colloidal systems, but albumin can form a matrix around the particles, which can protect them during transport in blood.

Example of Preparation of a Co-Colloidal Dispersion Comprising Anti-Cancer Drugs

Docetaxel, azacytidine and thalidomide are pharmacologically-active substances, known for their poor solubility in water and known for their therapeutic activity in certain types of cancer. Co-colloidal dispersions have been prepared according to the above-mentioned process and stored during one week at room temperature. The average particle size has been measured by DLS according to the above-mentioned method.

The following table summarizes these data.

| | | | Mean measures |
|---|---|---|---|
| | | | of particles |
| | | | by DLS after |
| | | | 1 week at |
| Example | Host molecule | Invited molecule | room |
| No. | (final concentration) | (final concentration) | temperature |
| 13 | C4S (5 mg/l) | Docetaxel (5 mg/l) | 221 nm |
| 14 | C6S (5 mg/ml) | Thalidomide (5 mg/l) | 112 nm |

Anti-Cancer Properties of Docetaxel Formulated as Co-Colloids

Swiss nude mice are irradiated and receive a subcutaneous injection of Calu-6 cells. Calu-6 cells are human lung tumor cells.

When the tumor reaches a volume of 100 to 200 mm³, the animals are randomly allocated to treatment groups. According to their group, the animals receive a series of intravenous injections of either placebo or docetaxel formulated under the form of a co-colloidal dispersion at concentrations ranging from 0.01% to 1%. The animals are examined daily for clinical signs and the tumor volume is measured. A satisfactory anti-cancer effect was observed with the treatment with docetaxel formulated under the form of co-colloids.

Application of Thalidomide Formulated as Co-Colloids to Treatment of AMD

Rabbits (strain: Fauve de Bourgogne) are used to induce a choroidal neovascularization. Burns of approximately 75 μm area are induced in the right eye of the animals with an argon laser at 532 nm applied for 0.1 second with a 150 mW intensity and using a Viridis photocoagulator (Quante Medical) around the optical disc and between the main vessel branches.

These burns induce the formation of neovessels that are measured by periodical opthalmological examinations. The animals are randomly allocated to treatment groups. According to their group, the animals receive an intravitreal injection of placebo or thalidomide formulated under the form of co-colloids at concentrations ranging from 0.01% to 1%. A satisfactory inhibitory effect of neovessels development is observed in the animals treated with thalidomide formulated under the form of co-colloids.

1. Co-colloidal dispersion in an aqueous medium of at least one supramolecular amphiphilic complex in which the at least one supramolecular amphiphilic complex comprises at least one hydrophilic molecule and at least one hydrophobic molecule associated by non-covalent bonds.

2. Co-colloidal dispersion according to claim 1, wherein the at least one hydrophilic molecule is chosen from hydrosoluble calixarenes.

3. Co-colloidal dispersion according to claim 1, wherein the at least one hydrophilic molecule is chosen from anionic hydrosoluble calixarenes.

4. Co-colloidal dispersion according to claim 1, wherein the at least one hydrophilic molecule of is chosen from parasulfonato calixarenes.

5. Co-colloidal dispersion according to claim 1, wherein the at least one hydrophobic molecule is chosen from molecules of pharmaceutical interest and cosmetic substances.

6. Co-colloidal dispersion according to claim 5, wherein the at least one hydrophobic molecule is chosen from substances that are active on the peripheral system, substances that are active on the central nervous system, substances that are active on renal function, substances that are active on cardiovascular function, substances that are active on gastro-intestinal function, substances that are active on blood function, substances that are active on immune function, substances that are active on hormonal function, substances that are active on genital function, substances that are active on reproductive functions, anti-inflammatory substances, anti-parasitic substances, antibiotics, anticancer substances, antidotes, vitamins, substances for parenteral nutrition, substances for dermatologic use, substances for topical use, and substances for ophthalmologic use.

7. A process for dispersion in an aqueous medium of at least one hydrophobic molecule of pharmaceutical interest comprising forming at least one supramolecular amphiphilic complex between the at least one hydrophobic molecule and at least one hydrophilic molecule.

8. A process for dispersion in an aqueous medium of at least one hydrophobic cosmetic substance comprising forming at least one supramolecular amphiphilic complex between the at least one substance and at least one hydrophilic molecule.

9. A process for preparing a co-colloidal dispersion according to claim 1, comprising
adding a composition comprising, in an organic solvent, at least one anionic hydrosoluble calixarene to a composition comprising, in an organic solvent, at least one hydrophobic molecule,
adding an aqueous solvent, and
eliminating the organic solvent.

10. Anti-cancer drugs in a form of a co-colloidal dispersion in an aqueous medium of at least one amphiphilic complex comprising at least one anionic hydrosoluble calixarene and at least one taxane.

11. Anti-cancer drugs according to claim 10, wherein the at least one taxane is docetaxel.

12. Anti-cancer drugs according to claim 11, in a form for use by the intravenous route for the treatment of cancer diseases.

13. Pharmaceutical drugs in a form of a co-colloidal dispersion in an aqueous medium of at least one amphiphilic complex comprising at least one anionic hydrosoluble calixarene and thalidomide.

14. Pharmaceutical drugs according to claim 13, in a form for use by the intravitreal route for the treatment of age-related macular degeneration.
